(19) United States
(12) Patent Application Publication
(10) Pub. No.: US 2025/0177468 A1
(43) Pub. Date: Jun. 5, 2025

(54) METHOD FOR TREATING CONDITIONS RELATED TO METABOLIC ARTHRITIS

(71) Applicant: HONGLI LEI, Corona, CA (US)

(72) Inventor: HONGLI LEI, Corona, CA (US)

(21) Appl. No.: 19/051,634

(22) Filed: Feb. 12, 2025

(51) Int. Cl. A61K 36/72; A61K 31/05; A61K 36/068; A61K 36/71; A61K 36/736; A61K 36/82; A61K 36/87; A61P 19/06

(52) U.S. Cl. CPC A61K 36/72; A61K 31/05; A61K 36/068; A61K 36/71; A61K 36/736; A61K 36/82; A61K 36/87; A61P 19/06

(57) ABSTRACT

A plant extract composition for treating metabolic arthritis (gout) in a subject includes 18%-22% of trans-resveratrol by weight of a total weight of plant extract composition; 24%-28% of hovenia dulcis fruit extract by weight of the total weight of plant extract composition; 11%-15% of China vine tea by weight of the total weight of plant extract composition; 10%-14% of tart cherry extract by weight of the total weight of plant extract composition; 10%-14% of green tea extract by weight of the total weight of the plant extract composition; 6%-10% of grapevine extract by weight of the total weight of the plant extract composition; and 6%-10% of Cordyceps militaris extract by weight of the total weight of the plant extract composition.

### FIELD OF THE DISCLOSURE

The present disclosure relates to a composition and methods for treating and preventing gout (metabolic arthritis). Such composition and methods may be used to treat a subject suffering from gout or to prevent the occurrence of the same in an at-risk subject.

### BACKGROUND OF THE DISCLOSURE

Generally, gout is a form of acute arthritis that causes severe joint pain and swelling. Gouty arthritis accounted for an estimated 3.9 million outpatient visits in the United States in 2002. Unlike other rheumatic diseases, the etiology of gout is well characterized, its pathophysiology is well understood, and the disease is easily diagnosed. For many patients, therapy with nonsteroidal anti-inflammatory drugs (NSAIDs) or corticosteroids for acute attacks and prevention of recurrence with agents that lower the serum uric acid levels are highly effective. However, these therapies are not sufficient for many patients with acute, chronic, or refractory gout due to their lack of adequate clinical efficacy, associated toxicities, or because of co-morbid diseases.

Known methods for treating gout include the use of uric acid synthesis inhibitors to inhibit the accumulation of uric acid in the body and the use of uric acid excretion promoters to accelerate the rapid excretion of uric acid accumulated in the body. Allopurinol is an example of a uric acid synthesis inhibitor.

In addition, the daily dosing of injectable medications is generally undesirable and may result in problems with patient compliance, thereby decreasing the effectiveness of this treatment modality/or limiting its desirability. Thus, there remains a need for effective means to treat gout, particularly treatment compositions and methods that do not require frequent (e.g., daily) injections. All referenced patents, applications, and literature are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply. The disclosed embodiments may seek to satisfy one or more of the above-mentioned desires. Although the present embodiments may obviate one or more of the above-mentioned desires, it should be understood that some aspects of the embodiments might not necessarily obviate them.

### BRIEF SUMMARY OF THE DISCLOSURE

In a general implementation, a method for treating metabolic arthritis in a subject, comprising:
orally administering to the subject a therapeutically effective amount of trans-resveratrol of 75 milligrams per day;
orally administering to the subject a therapeutically effective amount of hovenia dulcis fruit extract of 100 milligrams per day;
orally administering to the subject a therapeutically effective amount of China vine tea of 50 milligrams per day;
orally administering to the subject a therapeutically effective amount of tart cherry extract of 45 milligrams per day;
orally administering to the subject a therapeutically effective amount of green tea extract of 45 milligrams per day;
orally administering to the subject a therapeutically effective amount of grapevine extract of 30 milligrams per day; and
orally administering to the subject a therapeutically effective amount of Cordyceps militaris extract of 30 milligrams per day.

In another aspect combinable with the general implementation, the steps of administering to the subject the therapeutically effective amount of trans-resveratrol, hovenia dulcis fruit extract, China vine tea, tart cherry extract, green tea extract, grape vine extract, and Cordyceps militaris extract may comprise administering a daily dose once per day.

Among the many possible implementations of the steps of administering to the subject the therapeutically effective amount of trans-resveratrol, hovenia dulcis fruit extract, China vine tea, tart cherry extract, green tea extract, grape vine extract, and Cordyceps militaris extract may comprise steps of administering a daily dose once per day for 40 days.

Further, it is contemplated that treatment decreases serum uric acid produced from the subject's body and relieves pain within 15 days.

In the alternative, the trans-resveratrol, the hovenia dulcis fruit extract, the China vine tea, the tart cherry extract, the green tea extract, the grape vine extract, and the Cordyceps militaris extract are orally administered in a powder unit dose.

It is still further contemplated that the subject is a human.

In another aspect combinable with the general implementation, metabolic arthritis comprises asymptomatic gout, acute gout, and tophaceous gout.

Another aspect of the embodiment is directed to methods of treating metabolic arthritis in a subject that may comprise:
orally administering to the subject a therapeutically effective amount of plant extracts per day; wherein
the plant extracts consist of:
75 milligrams of trans-resveratrol;
100 milligrams of hovenia dulcis fruit extract;
50 milligrams of China vine tea;
45 milligrams of tart cherry extract;
45 milligrams of green tea extract;
30 milligrams of grapevine extract; and
30 milligrams of Cordyceps militaris extract.

In another aspect combinable with the general implementation, the steps of administering to the subject the therapeutically effective amount of plant extracts comprise administering a daily dose once per day.

In another aspect combinable with the general implementation, the steps of administering to the subject the therapeutically effective amount of plant extracts comprise administering a daily dose once for 40 days.

In another aspect combinable with the general implementation, the treatment decreases serum uric acid produced from the subject's body and relieves pain within 15 days.

In another aspect combinable with the general implementation, metabolic arthritis comprises asymptomatic gout, acute gout, and tophaceous gout.

In another aspect that is combinable with the general implementation, the subject is human.

In another aspect combinable with the general implementation, the trans-resveratrol, the hovenia dulcis fruit extract, the China vine tea, the tart cherry extract, the green tea extract, the grape vine extract, and the Cordyceps militaris extract are orally administered in a powder unit dose.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above and below as acting in certain combinations and even initially claimed as such, one or more features from a claimed combination can in some cases be excised from the combination, and the claimed combination may be directed to a subcombination or variation of a subcombination.

A number of implementations have been described. Nevertheless, it will be understood that various modifications may be made without departing from the spirit and scope of the disclosure. For example, example operations, methods, or processes described herein may include more steps or fewer steps than those described. Further, the steps in such example operations, methods, or processes may be performed in different successions than that described or illustrated in the figures. Accordingly, other implementations are within the scope of the following claims.

The details of one or more implementations of the subject matter described in this disclosure are set forth in the accompanying drawings and the description below. Other features, aspects, and advantages of the subject matter will become apparent from the description, the drawings, and the claims.

### BRIEF DESCRIPTION OF THE DRAWINGS

It should be noted that the drawing figures may be in simplified form and might not be to precise scale. In reference to the disclosure herein, for purposes of convenience and clarity only, directional terms such as top, bottom, left, right, up, down, over, above, below, beneath, rear, front, distal, and proximal are used with respect to the accompanying drawings. Such directional terms should not be construed to limit the scope of the embodiment in any manner.

FIG. 1 is a schematic diagram showing a method for treating metabolic arthritis in a subject according to an aspect of the embodiments.

FIG. 2 is another schematic diagram showing further steps of the method for treating metabolic arthritis in the subject according to an aspect of the embodiments.

FIG. 3 illustrates the experimental results of treating the plant extracts on zebrafishes to determine the mortality rate according to an aspect of the embodiments.

FIG. 4 illustrates experimental results on assisting in lowering uric acid according to an aspect of the embodiments.

FIG. 5 illustrates the fluorescence intensity of uric acid in zebrafish after treatment with plant extracts according to an aspect of the embodiments.

### DETAILED DESCRIPTION OF THE EMBODIMENTS

The different aspects of the various embodiments can now be better understood by turning to the following detailed description of the embodiments, which are presented as illustrated examples of the embodiments defined in the claims. It is expressly understood that the embodiments as defined by the claims may be broader than the illustrated embodiments described below.

The term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

It shall be understood that the term “means,” as used herein, shall be given its broadest possible interpretation in accordance with 35 U.S.C., Section 112(f). Accordingly, a claim incorporating the term “means” shall cover all structures, materials, or acts set forth herein, and all of the equivalents thereof. Further, the structures, materials or acts and the equivalents thereof shall include all those described in the summary of the invention, brief description of the drawings, detailed description, abstract, and claims themselves.

Unless defined otherwise, all technical and position terms used herein have the same meaning as commonly understood by one of ordinary skill in the art to which the invention pertains. Although many methods and materials similar, modified, or equivalent to those described herein can be used in the practice of the present invention without undue experimentation, the preferred materials and methods are described herein. In describing and claiming the present invention, the following terminology will be used in accordance with the definitions set out below.

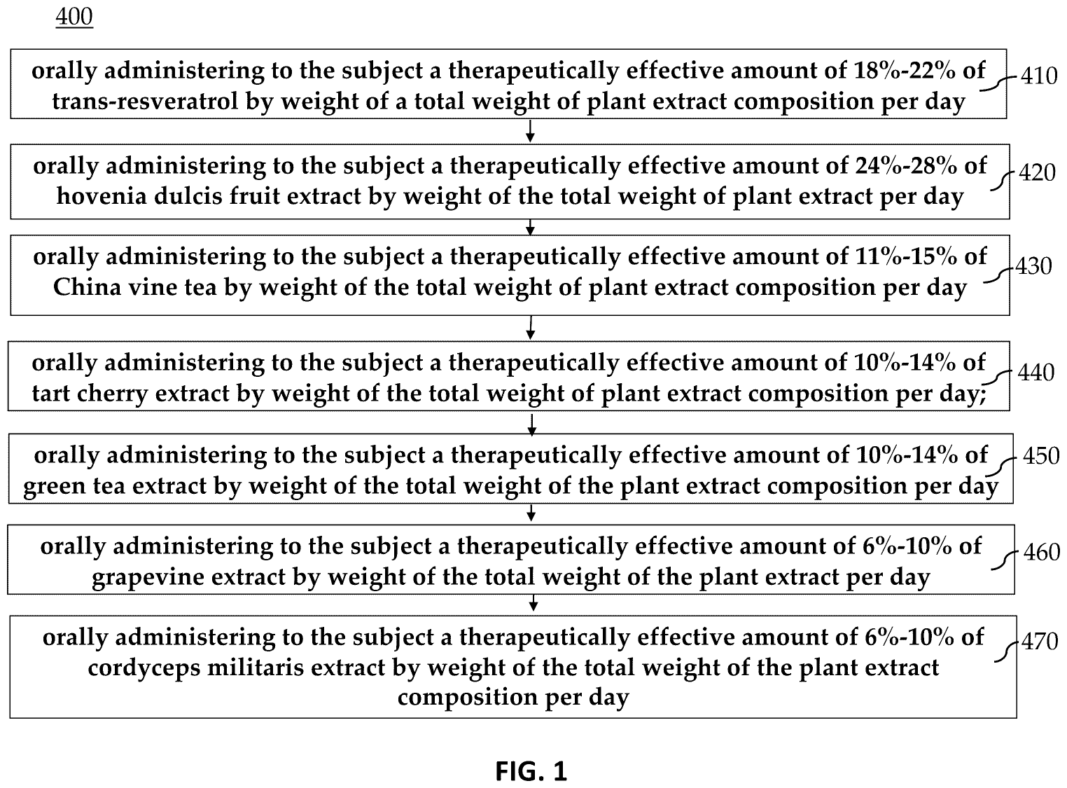

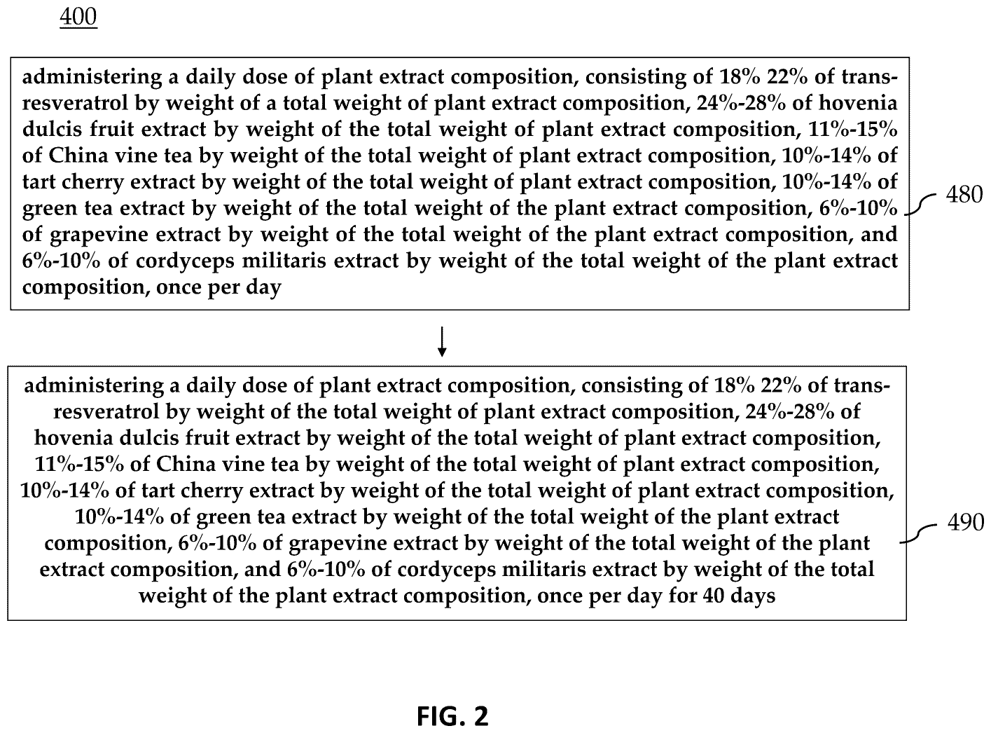

FIGS. 1-2 generally depicts a method for treating metabolic arthritis 400 in a subject according to an aspect of the embodiments.

Referring to FIG. 1, the method for treating metabolic arthritis 400 in the subject may comprise: orally administering to the subject a therapeutically effective amount of plant extract composition per day.

In some embodiments, the plant extract composition consists of:
18%-22% of trans-resveratrol by weight of a total weight of plant extract composition;
24%-28% of hovenia dulcis fruit extract by weight of the total weight of plant extract composition;
11%-15% of China vine tea by weight of the total weight of plant extract composition;
10%-14% of tart cherry extract by weight of the total weight of plant extract composition;
10%-14% of green tea extract by weight of the total weight of the plant extract composition;
6%-10% of grapevine extract by weight of the total weight of the plant extract composition; and
6%-10% of Cordyceps militaris extract by weight of the total weight of the plant extract composition.

Referring to FIG. 1, the method for treating metabolic arthritis 400 in the subject may comprise: orally administering to the subject a therapeutically effective amount of 18%-22% of trans-resveratrol by weight of a total weight of plant extract composition per day 410;
orally administering to the subject a therapeutically effective amount of 24%-28% of hovenia dulcis fruit extract by weight of the total weight of plant extract composition per day 420;
orally administering to the subject a therapeutically effective amount of 11%-15% of China vine tea by weight of the total weight of plant extract composition per day 430;
orally administering to the subject a therapeutically effective amount of 10%-14% of tart cherry extract by weight of the total weight of plant extract composition per day 440;
orally administering to the subject a therapeutically effective amount of 10%-14% of green tea extract by weight of the total weight of the plant extract composition per day 450;
orally administering to the subject a therapeutically effective amount of 6%-10% of grapevine extract by weight of the total weight of the plant extract per day 460; and
orally administering to the subject a therapeutically effective amount of 6%-10% of Cordyceps militaris extract by weight of the total weight of the plant extract composition per day 470.

In still some embodiments, the total weight of the plant extract composition may be 375 milligrams.

It should be noted that, preferably, in some embodiments, the plant extract composition may consist of: 20% of trans-resveratrol by weight of a total weight of plant extract composition, 27% of hovenia dulcis fruit extract by weight of the total weight of plant extract composition, 13% of China vine tea by weight of the total weight of plant extract composition, 12% of tart cherry extract by weight of the total weight of plant extract composition, 12% of green tea extract by weight of the total weight of the plant extract composition, 8% of grapevine extract by weight of the total weight of the plant extract composition, and 8% of Cordyceps militaris extract by weight of the total weight of the plant extract composition.

In still some embodiments, the therapeutically effective amount of trans-resveratrol may be 75 milligrams per day.

In still some embodiments, the therapeutically effective amount of hovenia dulcis fruit extract may be 100 milligrams per day;
In still some embodiments, the therapeutically effective amount of China vine tea may be 50 milligrams per day;
In still some embodiments, the therapeutically effective amount of tart cherry extract may be 45 milligrams per day;
In still some embodiments, the therapeutically effective amount of green tea extract may be 45 milligrams per day;
In still some embodiments, the therapeutically effective amount of grapevine extract may be 30 milligrams per day; and
In still some embodiments, the therapeutically effective amount of Cordyceps militaris extract may be 30 milligrams per day.

In some embodiments, the trans-resveratrol, the hovenia dulcis fruit extract, the China vine tea, the tart cherry extract, the green tea extract, the grape vine extract, and the Cordyceps militaris extract may be all plant extracts.

Continuing to FIG. 2, in some embodiments, the steps of orally administering to the subject the therapeutically effective amount of 18% 22% of trans-resveratrol by weight of the total weight of plant extract composition per day 410 may comprise steps of:
administering a daily dose of 18% 22% of trans-resveratrol by weight of the total weight of plant extract composition once per day 480.

In some embodiments, the steps of administering to the subject the therapeutically effective amount of 18% 22% of trans-resveratrol by weight of the total weight of plant extract composition once per day 480 may comprise the steps of:
administering a daily dose of 18% 22% of trans-resveratrol by weight of the total weight of plant extract composition once daily for 40 days 490.

In still some embodiments, the steps of orally administering to the subject the therapeutically effective amount of 24%-28% of hovenia dulcis fruit extract by weight of the total weight of plant extract composition per day 420 may comprise steps of:
administering a daily dose of hovenia dulcis fruit extract once per day 480.

In still some embodiments, the steps of orally administering to the subject the therapeutically effective amount of 24%-28% of hovenia dulcis fruit extract by weight of the total weight of plant extract composition once per day 480 may comprise steps of:
orally administering a daily dose of hovenia dulcis fruit extract once per day for 40 days 490.

In still some embodiments, the steps of orally administering to the subject the therapeutically effective amount of 11%-15% of China vine tea by weight of the total weight of plant extract composition per day 430 may comprise steps of:
administering a daily dose of China vine tea once per day 480.

In still some embodiments, the steps of administering to the subject the therapeutically effective amount of 11%-15% of China vine tea by weight of the total weight of plant extract composition once per day 480 may comprise steps of:
administering a daily dose of China vine tea once per day for 40 days 490.

In still some embodiments, the steps of orally administering to the subject the therapeutically effective amount of 10%-14% of tart cherry extract by weight of the total weight of plant extract composition per day 440 may comprise steps of:
administering a daily dose of tart cherry extract once per day 480.

In still some embodiments, the steps of administering to the subject the therapeutically effective amount of tart cherry extract once per day 480 may comprise steps of:
administering a daily dose of tart cherry extract once per day for 40 days 490.

In still some embodiments, the steps of administering to the subject the therapeutically effective amount of 10%-14% of green tea extract by weight of the total weight of the plant extract composition per day 450 may comprise steps of:
administering a daily dose of the green tea extract once per day 480.

In still some embodiments, the steps of administering to the subject the therapeutically effective amount of the green tea extract once per day 480 may comprise steps of:
administering a daily dose of the green tea extract once per day for 40 days 490.

In still some embodiments, the steps of administering the therapeutically effective amount of 6%-10% of grapevine extract by weight of the total weight of the plant extract composition may comprise steps of:
administering a daily dose of the grapevine extract once per day 480.

In still some embodiments, the steps of administering the therapeutically effective amount of grapevine extract once per day 480 may comprise steps of:
administering a daily dose of the grapevine extract once per day for 40 days 490.

In still some embodiments, the steps of administering to the subject the therapeutically effective amount of 6%-10% of Cordyceps militaris extract by weight of the total weight of the plant extract composition per day 470 may comprise steps of:
administering a daily dose of Cordyceps militaris extract once per day 480.

In still some embodiments, the steps of administering to the subject the therapeutically effective amount of Cordyceps militaris extract once per day 480 may comprise steps of:
administering a daily dose of Cordyceps militaris extract once per day for 40 days 490.

According to the above steps, it should be noted that, in some embodiments, treatment results in decreased serum uric acid produced from the human body and relieving pain within 15 days.

In some embodiments, metabolic arthritis may comprise asymptomatic gout, acute gout, and tophaceous gout.

In still some embodiments, the trans-resveratrol, the hovenia dulcis fruit extract, the China vine tea, the tart cherry extract, the green tea extract, the grape vine extract, and the Cordyceps militaris extract may be orally administered in a powder unit dose.

In still some embodiments, the powder unit dose may form a capsule, and in certain embodiments, it may be a tablet. Such tablets or capsules may not include additional agents such as binders, lubricants, diluents, etc. In other words, the powder unit dose consists of the trans-resveratrol, the hovenia dulcis fruit extract, the China vine tea, the tart cherry extract, the green tea extract, the grape vine extract, and the Cordyceps militaris extract.

As shown in further detail in FIGS. 1-2, the method for treating metabolic arthritis the subject may comprise steps of:
orally administering to the subject a therapeutically effective amount of plant extracts per day; wherein
the plant extracts consist of:
75 milligrams of trans-resveratrol;
100 milligrams of hovenia dulcis fruit extract;
50 milligrams of China vine tea;
45 milligrams of tart cherry extract;
45 milligrams of green tea extract;
30 milligrams of grapevine extract; and
30 milligrams of Cordyceps militaris extract.

In some embodiments, the plant extract may form a powder unit dose which may form a capsule, and in certain embodiments, it may be a tablet. Such tablets or capsules may not include additional agents such as binders, lubricants, diluents, etc. In other words, the powder unit dose consists of the trans-resveratrol, the hovenia dulcis fruit extract, the China vine tea, the tart cherry extract, the green tea extract, the grape vine extract, and the Cordyceps militaris extract.

In some embodiments, the step of orally administering to the subject the therapeutically effective amount of plant extracts per day may comprise steps of:
administering to the subject the therapeutically effective amount of trans-resveratrol of 75 milligrams per day; and
administering a daily dose of the trans-resveratrol of 75 milligrams once per day.

In some embodiments, the step of orally administering to the subject the therapeutically effective amount of plant extracts per day may comprise steps of:
administering to the subject the therapeutically effective amount of trans-resveratrol of 75 milligrams once per day; and
administering a daily dose of the trans-resveratrol of 75 milligrams once daily for 40 days.

In some embodiments, the step of orally administering to the subject the therapeutically effective amount of plant extracts per day may comprise steps of:
administering to the subject the therapeutically effective amount of the hovenia dulcis fruit extract of 100 milligrams per day; and
administering a daily dose of hovenia dulcis fruit extract of 100 milligrams once per day.

In some embodiments, the step of orally administering to the subject the therapeutically effective amount of plant extracts per day may comprise steps of:
administering to the subject the therapeutically effective amount of the hovenia dulcis fruit extract of 100 milligrams once per day; and
administering a daily dose of hovenia dulcis fruit extract of 100 milligrams once per day for 40 days.

In some embodiments, the step of orally administering to the subject the therapeutically effective amount of plant extracts per day may comprise steps of:
administering to the subject the therapeutically effective amount of China vine tea of 50 milligrams per day; and
administering a daily dose of China vine tea of 50 milligrams once per day.

In some embodiments, the step of orally administering to the subject the therapeutically effective amount of plant extracts per day may comprise steps of:
administering to the subject the therapeutically effective amount of China vine tea of 50 milligrams once per day; and
administering a daily dose of China vine tea of 50 milligrams once per day for 40 days.

In some embodiments, the step of orally administering to the subject the therapeutically effective amount of plant extracts per day may comprise steps of:
administering to the subject the therapeutically effective amount of tart cherry extract of 45 milligrams per day; and administering a daily dose of tart cherry extract of 45 milligrams once per day.

In some embodiments, the step of orally administering to the subject the therapeutically effective amount of plant extracts per day may comprise steps of:
administering to the subject the therapeutically effective amount of tart cherry extract of 45 milligrams once per day; and
administering a daily dose of tart cherry extract of 45 milligrams once per day for 40 days.

In some embodiments, the step of orally administering to the subject the therapeutically effective amount of plant extracts per day may comprise steps of:
administering to the subject the therapeutically effective amount of green tea extract of 45 milligrams per day; and
administering a daily dose of the green tea extract of 45 milligrams once per day.

In some embodiments, the step of orally administering to the subject the therapeutically effective amount of plant extracts per day may comprise steps of:
administering to the subject the therapeutically effective amount of green tea extract of 45 milligrams once per day; and
administering a daily dose of the green tea extract of 45 milligrams once per day for 40 days.

In some embodiments, the step of orally administering to the subject the therapeutically effective amount of plant extracts per day may comprise steps of:
administering the therapeutically effective amount of grapevine extract of 30 milligrams per day; and
administering a daily dose of the grapevine extract of 30 milligrams once per day.

In some embodiments, the step of orally administering to the subject the therapeutically effective amount of plant extracts per day may comprise steps of:
administering the therapeutically effective amount of grapevine extract of 30 milligrams once per day; and
administering a daily dose of the grapevine extract of 30 milligrams once per day for 40 days.

In some embodiments, the step of orally administering to the subject the therapeutically effective amount of plant extracts per day may comprise steps of:
administering to the subject the therapeutically effective amount of Cordyceps militaris extract of 30 milligrams per day; and
administering a daily dose of Cordyceps militaris extract of 30 milligrams once per day.

In some embodiments, the step of orally administering to the subject the therapeutically effective amount of plant extracts per day may comprise steps of:
administering to the subject the therapeutically effective amount of Cordyceps militaris extract of 30 milligrams once per day; and
administering a daily dose of Cordyceps militaris extract of 30 milligrams once per day for 40 days.

According to the above steps, it should be noted that, in some embodiments, treatment results in decreased serum uric acid produced from the human body and relieves pain within 15 days.

In some embodiments, metabolic arthritis may comprise asymptomatic gout, acute gout, and tophaceous gout.

In still some embodiments, the trans-resveratrol, the hovenia dulcis fruit extract, the China vine tea, the tart cherry extract, the green tea extract, the grape vine extract, and the Cordyceps militaris extract may be orally administered in a powder unit dose.

A plant extract composition for treating metabolic arthritis (gout) in the subject, comprising:
18%-22% of trans-resveratrol by weight of a total weight of plant extract composition;
24%-28% of hovenia dulcis fruit extract by weight of the total weight of plant extract composition;
11%-15% of China vine tea by weight of the total weight of plant extract composition;
10%-14% of tart cherry extract by weight of the total weight of plant extract composition;
10%-14% of green tea extract by weight of the total weight of the plant extract composition;
6%-10% of grapevine extract by weight of the total weight of the plant extract composition; and
6%-10% of Cordyceps militaris extract by weight of the total weight of the plant extract composition.

In some embodiments, the plant extract composition is orally administered to the subject once per day.

In some embodiments, the total weight of the plant extract composition may be 375 milligrams.

In still some embodiments, the plant extract composition may comprise 20% of trans-resveratrol by weight of a total weight of plant extract, 27% of hovenia dulcis fruit extract by weight of the total weight of plant extract, 13% of China vine tea by weight of the total weight of plant extract, 12% of tart cherry extract by weight of the total weight of plant extract, 12% of green tea extract by weight of the total weight of the plant extract, 8% of grapevine extract by weight of the total weight of the plant extract, and 8% of Cordyceps militaris extract by weight of the total weight of the plant extract.

In some embodiments, the plant extract composition may be orally administered to the subject once daily for 40 days.

In still some embodiments, the treatment results in decreased serum uric acid produced from the subject's body and relieved pain within 15 days.

In still some embodiments, the plant extract composition may be orally administered to the subject in a powder unit dose.

In still some embodiments, the subject is a human.

In still some embodiments, the metabolic arthritis comprises asymptomatic gout, acute gout, and tophaceous gout.

### 1. Example 1

Effects of lowering uric acid on zebrafish (Danio rerio)

### 1.1. Sample Preparation

The plant extract is mixed with standard diluted water to form a 20.0 mg/mL mother solution.

Positive Control Group: allopurinol, white powder, lot number MKBZ0649V, Sigma-Aldrich (Shanghai) Trading Co., Ltd., stored in a cool and dark place and prepared a 13.6 mg/mL mother liquor having DMSO and stored at the temperature of −20° C.

### 1.2. Test Animals

Zebrafishes are all raised in fish farming water at the temperature of 28° C. (water quality: 200 mg of instant sea salt is added to every 1 L of reverse osmosis water, and the conductivity is 450˜550 μS/cm; pH is 6.5˜8.5, hardness is 50˜100 mg/L CaCO3). The license number for the test animals is SYXK (Zhejiang) 2012-0171, with the requirements of the international AAALAC certification (certification number: 001458).

Wild-type AB strains of zebrafish are propagated in natural pairs. The age of Zebrafishes are 5 days after fertilization (5 dpf) and are used to determine the maximum tolerable concentration (MTC) of lowering uric acid efficacy and the evaluation of efficacy of lowering uric acid for plant extracts.

### 1.3. Instruments and Reagents

Dissecting microscope (SZX7, OLYMPUS, Japan); CCD camera (VertA1, Shanghai Tusen Vision Technology Co., Ltd., China); Multi-mode microplate reader (SPARK, TECAN, Switzerland); Precision electronic balance (CP214, OHAUS, USA); 6-well plates (Nest Biotech, China); 96-well plates (Nest Biotech, China).

Amplex™ Red Uric Acid/Uricase Assay Kit (lot number 2077764, Thermo Fisher Scientific, USA); Potassium oxazinate (batch number is C1808048, Shanghai Aladdin Biochemical Technology Co., Ltd., China); xanthine sodium salt (lot number SLBV3159, Sigma, China); Dimethyl sulfoxide (DMSO, lot number BCBZ1685, Sigma, USA).

### 2. Detection Method

### 2.1. MTC (Maximum Tolerable Concentration) Determination

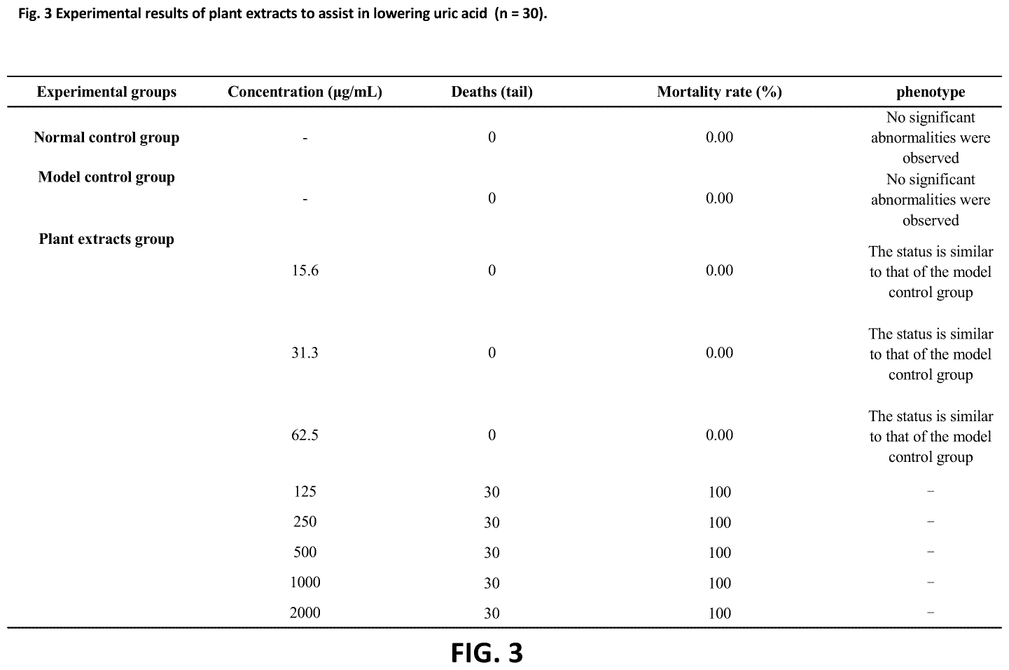

30 pieces of the 5 dpf wild-type AB strains of zebrafish are randomly selected, placed in the 6-well plates, and treated as an experimental group. Plant extracts are prepared as water-solution (see FIG. 3 for concentration) for the normal control group and model control group, with a capacity of 3 mL per well. Except for the normal control group, potassium oxyazinate and xanthine sodium salt are mixed in water for the rest of experimental groups to establish a high uric acid zebrafish model. After 1 day of treatment at 28° C., the MTC for zebrafish in the plant extracts group is determined.

### 2.2. Efficacy Evaluation of Lowering Uric Acid

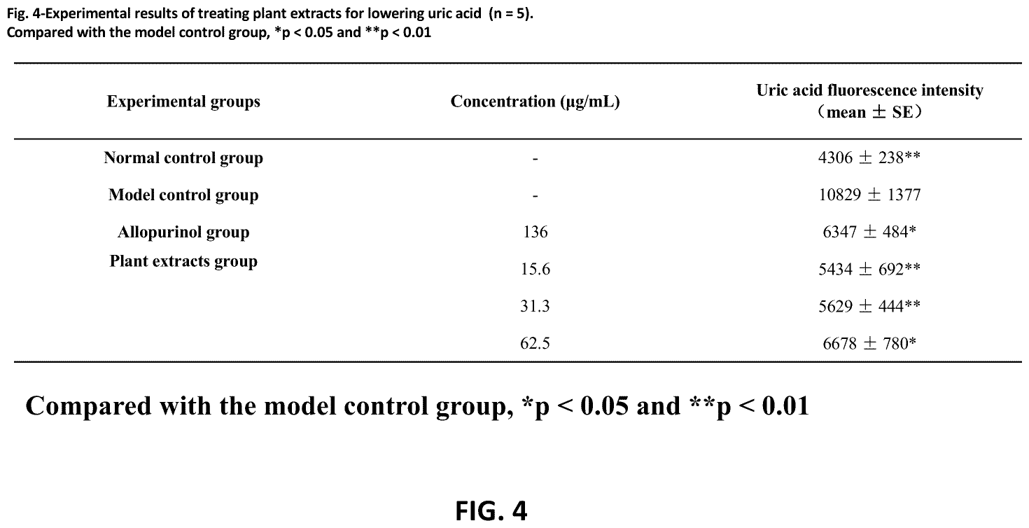

30 pieces of 5 dpf wild-type AB strain of zebrafish are randomly selected and placed in 6-well plates and are treated in each well as the experimental groups. Samples of plant extracts (see FIG. 4) are mixed with water to form the plant extracts group, and the allopurinol group is concentrated at a concentration of 136 g/mL, and the normal control group and the model control group are set up at the same time, with a capacity of 3 mL per well. Except for the normal control group, potassium oxyazinate, and xanthine sodium salt are dissolved in water in the rest of the experimental groups to establish a high uric acid zebrafish model. After 1 day of treatment at 28° C., the uric acid fluorescence detection kit detects the uric acid content in zebrafish. Multi-function microplate reader software analyzes the uric acid fluorescence intensity (S) in zebrafish. The efficacy of plant extract samples is evaluated by analyzing the statistical results of this index. The results of statistical processing are expressed as mean±SE. Statistical analysis is performed with SPSS26.0 software; p<0.05 indicates that the difference is statistically significant.

### 3. Test Results

### 3.1. MTC (Maximum Tolerable Concentration)

Under the conditions of this experiment, the MTC of plant extracts for zebrafish is 62.5 g/mL (see FIG. 3 for details).

### 3.2. Effects for Lowering Uric Acid

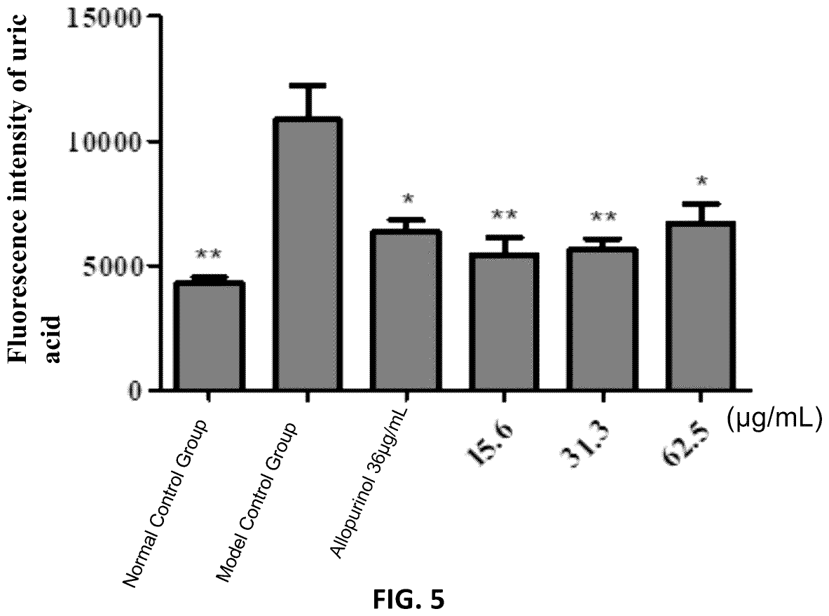

Under the conditions of this experiment, the samples of the plant extracts have obvious effects for lowering uric acid (See FIG. 4). As shown in FIG. 5. the Fluorescence intensity of uric acid in zebrafish after treating with plant extracts is compared with the model control group and the normal control group, *p<0.05 and **p<0.01.

Many alterations and modifications may be made by those having ordinary skill in the art without departing from the spirit and scope of the disclosed embodiments. Therefore, it must be understood that the illustrated embodiments have been set forth only for the purposes of example and that it should not be taken as limiting the embodiments as defined by the following claims. For example, even though the elements of a claim are set forth below in a certain combination, it must be expressly understood that the embodiment includes other combinations of fewer, more, or different elements, which are disclosed herein even when not initially claimed in such combinations.

Thus, specific embodiments and applications of a method for treating metabolic arthritis in the subject have been disclosed. It should be apparent, however, to those skilled in the art that many more modifications besides those already described are possible without departing from the disclosed concepts herein. Therefore, the disclosed embodiments are not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Insubstantial changes from the claimed subject matter as viewed by a person with ordinary skill in the art, now known or later devised, are expressly contemplated as being equivalent within the scope of the claims. Therefore, obvious substitutions now or later known to one with ordinary skill in the art are defined to be within the scope of the defined elements. The claims are thus to be understood to include what is specifically illustrated and described above, what is conceptually equivalent, what can be obviously substituted and also what essentially incorporates the essential idea of the embodiments. In addition, where the specification and claims refer to at least one of something selected from the group consisting of A, B, C . . . and N, the text should be interpreted as requiring at least one element from the group which includes N, not A plus N, or B plus N, etc.

The words used in this specification to describe the various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The definitions of the words or elements of the following claims therefore include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

What is claimed is:

1. A plant extract composition for treating metabolic arthritis (gout) in a subject, consisting of:
18%-22% of trans-resveratrol by weight of a total weight of plant extract composition;
24%-28% of hovenia dulcis fruit extract by weight of the total weight of plant extract composition;
11%-15% of China vine tea by weight of the total weight of plant extract composition;
10%-14% of tart cherry extract by weight of the total weight of plant extract composition;
10%-14% of green tea extract by weight of the total weight of the plant extract composition;
6%-10% of grapevine extract by weight of the total weight of the plant extract composition; and
6%-10% of Cordyceps militaris extract by weight of the total weight of the plant extract composition.

2. The plant extract composition of claim 1, wherein the plant extract composition is orally administered to the subject once per day.

3. The plant extract composition of claim 1, wherein the total weight of the plant extract composition is 375 milligrams.

4. The plant extract composition of claim 1, wherein the composition comprises 20% of trans-resveratrol by weight of the total weight of plant extract, 27% of hovenia dulcis fruit extract by weight of the total weight of plant extract, 13% of China vine tea by weight of the total weight of plant extract, 12% of tart cherry extract by weight of the total weight of plant extract, 12% of green tea extract by weight of the total weight of the plant extract, 8% of grapevine extract by weight of the total weight of the plant extract, and 8% of Cordyceps militaris extract by weight of the total weight of the plant extract.

5. The plant extract composition of claim 1, wherein the plant extract composition is orally administered to the subject once per day for 40 days.

6. The plant extract composition of claim 1, wherein treatment results in decreased serum uric acid produced from the subject's body and relieved pain within 15 days.

7. The plant extract composition of claim 1, wherein the plant extract composition is orally administered to the subject in a powder unit dose.

8. The plant extract composition of claim 1, wherein the subject is a human.

9. The plant extract composition of claim 1, wherein the metabolic arthritis comprises asymptomatic gout, acute gout, and tophaceous gout.

10. A method for treating metabolic arthritis (gout) in a subject, comprising:
orally administering to the subject a therapeutically effective amount of plant extract composition per day; wherein
the plant extract composition consists of:
18%-22% of trans-resveratrol by weight of a total weight of plant extract composition;
24%-28% of hovenia dulcis fruit extract by weight of the total weight of plant extract composition;
11%-15% of China vine tea by weight of the total weight of plant extract composition;
10%-14% of tart cherry extract by weight of the total weight of plant extract composition;
10%-14% of green tea extract by weight of the total weight of the plant extract composition;
6%-10% of grapevine extract by weight of the total weight of the plant extract composition; and
6%-10% of Cordyceps militaris extract by weight of the total weight of the plant extract composition.

11. The method of claim 10, wherein the total weight of the plant extract composition is 375 milligrams.

12. The method of claim 10, wherein the plant extract composition comprises 20% of trans-resveratrol by weight of a total weight of plant extract composition, 27% of hovenia dulcis fruit extract by weight of the total weight of plant extract composition, 13% of China vine tea by weight of the total weight of plant extract composition, 12% of tart cherry extract by weight of the total weight of plant extract composition, 12% of green tea extract by weight of the total weight of the plant extract composition, 8% of grapevine extract by weight of the total weight of the plant extract composition, and 8% of Cordyceps militaris extract by weight of the total weight of the plant extract composition.

13. The method of claim 10, wherein the steps of orally administering to the subject the therapeutically effective amount of plant extracts comprises administering a daily dose once per day.

14. The method of claim 10, wherein the steps of orally administering to the subject the therapeutically effective amount of plant extracts comprises administering a daily dose once per day for 40 days.

15. The method of claim 10, wherein treatment results in decreased serum uric acid produced from the subject's body and relieved pain within 15 days.

16. The method of claim 10, wherein the metabolic arthritis comprises asymptomatic gout, acute gout, and tophaceous gout.

17. The method of claim 10, wherein the subject is a human.

18. The method of claim 10, wherein the trans-resveratrol, the hovenia dulcis fruit extract, the China vine tea, the tart cherry extract, the green tea extract, the grape vine extract, and the Cordyceps militaris extract are orally administered in a powder unit dose.
